(19) United States
(12) Patent Application Publication
(10) Pub. No.: US 2004/0096923 A1
(43) Pub. Date: May 20, 2004

(54) Lipid phosphatase assays in disease and drug discovery

(75) Inventors: Beth E. Drees, Park City, UT (US); Paul O. Neilsen, Draper, UT (US); Angie M. Branch, West Jordan, UT (US); Amber Weipert, Draper, UT (US)

Correspondence Address: THORPE NORTH & WESTERN, LLP., 8180 SOUTH 700 EAST, SUITE 200, P.O. BOX 1219, SANDY, UT, 84070, US

(73) Assignee: ECHELON BIOSCIENCES INCORPORATED

(21) Appl. No.: 10/712,073

(22) Filed: Nov. 13, 2003

Related U.S. Application Data
(60) Provisional application No. 60/426,572, filed on Nov. 15, 2002.

(51) Int. Cl. G01N 33/53

(52) U.S. Cl. 435/7.92

(57) ABSTRACT

A lipid assay method, kit, and apparatus involving exposing a lipid detector protein containing a lipid recognition motif with a binding specificity for a product lipid of a lipid phosphatase, to a solution containing a substrate lipid of the lipid phosphatase; and determining whether the product lipid is present. The assay is preferably a competitive assay wherein the product lipid has a stronger affinity to the lipid detector protein than the substrate lipid. The lipid recognition motif is preferably a pleckstrin homology (PH) domain.

This application claims the benefit of U.S. Provisional Application No.60/426,572, filed on Nov. 15, 2002.

### BACKGROUND OF THE INVENTION

### Field of the Invention

The present invention relates to enzymes that dephosphorylate inositol lipids, including phosphatidylinositol (3,4,5) trisphosphate (PI(3,4,5)P₃) and other phosphotidylinositol phosphates (PIPₙs) in general, at the D-3, D-4, or D-5 positions of the inositol ring. More particularly, the present invention relates to detection methods, kits, and apparatuses for the detection of such enzymes.

Phosphoinositide phosphates are important second messengers in signaling pathways governing cellular proliferation, survival, morphology, and motility. There is described herein the development of assay methods for determination of phosphoinositide phosphatase activity. Lipid phosphatases and alterations in their activity levels, are implicated in a variety of signaling pathways that are important in regulation of insulin sensitivity and allergic and immune responses, and which are altered in carcinogenesis. Because signaling pathways involving these lipid modifying enzymes are often perturbed in the events leading to disease, particularly non-insulin dependent diabetes mellitus (NIDDM) and cancer, these assays have significant value as research and diagnostic tools as well as drug-screening platforms.

Although phosphatidylinositol phosphates (PIPs) are minor components of cellular membranes, PIP-dependent signaling pathways play central roles in the regulation of many cellular processes.(Martin, Ann. Rev. Cell Dev. Biol., 14:231-2614 (1998)) PIPs are distinctive among phospholipids in their ability to be quickly modified by phosphorylation or dephosphorylation of their inositol headgroups, and thus to act as second messengers which regulate site-specific signaling and assembly of membrane-associated protein complexes. Because the activity of PIP second messengers is determined by their phosphorylation state, the enzymes that act to modify these lipids are central to the correct execution of these signaling events.(Leslie, Chemical Reviews, 101:2365-2380 (2001)) PIP biosynthesis occurs through the interplay of lipid-specific kinases and phosphatases, as shown in FIG. 1. Many of these enzymes specifically modulate signaling by type I phosphoinositide 3-kinases (PI 3-Ks) to regulate cell growth and morphology. PI(3,4,5)P₃-regulated signaling is governed both by its formation by PI 3-K and by its conversion into diphosphates, such as PI(4,5)P₂ or PI(3,4)P₂. The activities of 3′ and 5′ phosphoinositide phosphatases, such as the enzymes PTEN and SHIP respectively, are important for this regulation. Myotubularin and related phosphatases remove the 3′ phosphate of PI(3)P to regenerate PI, and play a role in vesicular trafficking by modulating type III PI 3-Ks, which regulate trafficking via phosphorylation of PI. These enzymes, and alterations in their activity levels, are implicated in a variety of signaling pathways that are important in regulation of insulin sensitivity and allergic and immune responses, and which are altered in carcinogenesis. Table 1 summarizes the activity of the enzymes that are the focus of this application and their importance in disease.

TABLE 1
Selected Lipid phosphatases and their roles in disease.

| Enzyme | Activity | Importance in Disease |
|---|---|---|
| PTEN | 3′ phosphatase, | Tumor suppressor, mutated in many cancer types. |
| | Converts PI(3, 4, 5)P₃ to PI(4, 5)P₂ | Germline mutation associated with multiple |
| | | hamartoma syndromes, such as Cowden's disease. |
| | | Implicated in regulation of insulin signaling and |
| | | sensitivity, potential point of therapeutic intervention |
| | | for NIDDM. |
| SHIP1 | 5′ phosphatase, | Loss of activity associated with Chronic Myelogenous |
| | Converts PI(3, 4, 5)P₃ to PI(3, 4)P₂ | Leukemia. |
| | | Negative regulator of mast cell degranulation, defects |
| | | in SHIP activity may be associated with allergic |
| | | response. |
| SHIP 2 | 5′ phosphatase, | Implicated in regulation of insulin signaling and |
| | Converts PI(3, 4, 5)P₃ to PI(3, 4)P₂ | sensitivity, potential point of therapeutic intervention |
| | | for NIDDM. |
| PTPRQ | Broad specificity phosphatase. | Implicated in regulation of cell survival and |
| | Most active in removal of 3′ and | proliferation. |
| | 5′ phosphates from PI(3, 4, 5)P₃, | Overexpression inhibits proliferation of Glioblastoma |
| | PI(3, 5)P₂, and PI(4,5)P₂. | cells and leads to arrest and apoptosis. |
| Skeletal muscle | 5′ inositol phosphatase which | Like SHIP2, is implicated in regulation of insulin |
| and Kidney | acts on both PI(3, 4, 5)P₃ and | sensitivity and is a potential point of therapeutic |
| Specific Inositol | PI(3, 5)P₂. | intervention for NIDDM. |
| Phosphatase | | |
| (SKIP) | | |
| Myotubularin & | 3′ PI(3)P and PI(3, 5)P₂ | Mutated in X-linked myotubular myopathy and |
| MTMR2 | phosphatase | demyelinating Charcot-Marie-Tooth disease. |

The myotubularin family of 3′ lipid phosphatases act on PI(3)P and/or PI(3,5)P₂ as their substrates and play a role in vesicle trafficking and autophagy (Taylor, et al., Proc Natl Acad Sci USA, 97:8910-5. (2000)). PI(3)P is generated by the activity of type III PI 3-kinases, which have specific activity toward PI and have physiological roles distinct from type I PI 3-Ks, which act in growth regulation and signaling. PI(3)P is important for vesicle trafficking, and the myotubularins may be important in renewing the pool of PI needed for these processes to be in balance. There are at least ten members of this protein family in humans (Laporte, et al., Hum Mol Genet, 7:1703-12. (1998)). Myotubularin, encoded by the MTM1 gene, is inactivated by mutation in X-linked myotubular myopathy, a severe and fatal congenital disease(Laporte, et al., Nat Genet, 13:175-82. (1996), Blondeau, et al., Hum Mol Genet, 9:2223-9. (2000)). Studies of MTM1 knockout mice show that the onset of progressive myopathy results from a defect in structural maintenance of muscle rather than defective myogenesis(Buj-Bello, et al., Proc Natl Acad Sci USA, 99:15060-5. (2002)). Some patients with a rare form of Charcot-Marie-Tooth disease have truncations in a second family member, MTMR2, which results in loss of protein expression(Bolino, et al., Nat Genet, 25:17-9. (2000)). Characteristics of this disease, which most commonly is associated with mutations in other genes, are motor and sensory neuropathy with onset during infancy, with abnormal myelination and Schwann cell proliferation in peripheral nerves. Since the disease phenotypes associated with the myotubularins result from loss-of-function mutations, currently they are not thought to be targets for drug development. However, the methods disclosed in the present invention may have potential application for assaying the activity of these enzymes.

The 5′ lipid phosphatase SHIP1 (SH2-containing inositol phosphatase 1) acts as a negative regulator of cytokine signaling and immune cell activation and differentiation (Bolland, et al., Immunity, 8:509-16. (1998), Liu, et al., J Exp Med, 188:1333-42. (1998), Brauweiler, et al., J Exp Med, 191:1545-54. (2000), Brauweiler, et al., Immunol Rev, 176:69-74. (2000), Rohrschneider, et al., Genes Dev, 14:505-20. (2000)), and regulates differentiation and maintenance of hematopoetic cell lineages.(Liu, et al., Blood, 91:2753-9. (1998)). SHIP1 is a 5′ lipid phosphatase which converts PI(3,4,5)P₃ to PI(3,4)P₂.(Damen, et al., Proc Natl Acad Sci USA, 93:1689-93. (1996, Lioubin, et al., Genes Dev, 10:1084-95. (1996))SHIP1 is a 5′ lipid phosphatase which converts PI(3,4,5)P₃ to PI(3,4)P₂.(Damen, et al., Proc Natl Acad Sci USA, 93:1689-93. (1996, Lioubin, et al., Genes Dev, 10:1084-95. (1996)) Expression is restricted to cells of hematopoetic origin, including granulocytes, monocytes, and the lymphocytes, B and T cells.(Liu, et al., Blood, 91:2753-9. (1998)) Like PTEN, SHIP1 acts as a negative regulator of PI 3-K mediated signaling and activation of Akt/PKB.(Aman, et al., J Biol Chem, 273:33922-8. (1998), Liu, et al., Genes Dev, 13:786-91. (1999)) In response to cytokine stimulation or B and T cell receptor engagement, SHIP1 is tyrosine phosphorylated and translocated to the cytoskeleton via interaction with the Shc adaptor protein.(Damen, et al., Proc Natl Acad Sci USA, 93:1689-93. (1996), Liu, et al., Genes Dev, 13:786-91. (1999)). Ablation of SHIP1 in transgenic mice leads to chronic hyperplasia and increased proliferation and survival of hematopoetic cells in response to cytokine treatment (Helgason, et al., Genes Dev, 12:1610-20. (1998), Brauweiler, et al., J Exp Med, 191:1545-54. (2000)).

SHIP1-regulated signaling is important for modulation of allergic responses. IgE receptor ligation by an allergan leads to PI 3-K activation and eventual mast cell degranulation. SHIP1 hydrolysis of PI(3,4,5)P₃ modulates PI 3-K signaling to set the threshold for mast cell degranulation through its interaction with the immune inhibitory receptor FcγRIIB (Huber, et al., Proc Natl Acad Sci USA, 95:11330-5. (1998)). Mast cells derived from animals that carry a homozygous deletion of SHIP1 are significantly more prone to degranulation in response to IgE receptor ligation.(Huber, et al., Embo J, 17:7311-9. (1998)) A similar exaggerated IgE response and enhanced induction of mast cell degranulation is associated with clinical allergies and asthma. Thus, modulation of SHIP1 activity may be an avenue for therapeutic intervention in the treatment of allergies and other immune disorders.

The regulation of PI(3,4,5)P₃ levels is often defective in tumorigenesis.(Carpenter and Cantley, Biochim Biophys Acta, 1288:M11-6. (1996), Roymans and Slegers, Eur J Biochem, 268:487-98. (2001)) Elevated PI(3,4,5)P₃ levels contribute to cancer progression through constitutive activation of PKB/Akt.(Franke, et al., Cell, 81:727-36. (1995), King, et al., Mol Cell Biol, 17:4406-18. (1997)), which provides a cell survival signal that blocks apoptosis and promotes survival following growth factor withdrawal or detachment from the extracellular matrix.(Franke, et al., Cell, 88:435-7. (1997), Kim, et al., Faseb J, 15:1953-62. (2001)) Elevated levels of PI(3,4,5)P₃ can occur through amplification of PI 3-K gene expression, as is seen in some cancers (Shayesteh, et al., Nat Genet, 21:99-102. (1999), Ma, et al., Oncogene, 19:2739-44. (2000)), or through alterations in the activity of the lipid phosphatases which are responsible for modulating PI(3,4,5)P₃ levels.

PTEN (Phosphatase and Tensin Homolog deleted on Chromosome 10), also designated MMAC 1 (Mutated in Multiple Advanced Cancers), is a 3′ phosphoinositide phosphatase that converts PI(3,4,5)P₃ to PI(4,5)P₂. (Maehama and Dixon, J Biol Chem, 273:13375-8. (1998), Maehama and Dixon, Trends Cell Biol, 9:125-8. (1999)) By converting PI(3,4,5)P₃ to PI(4,5)P₂, PTEN acts as a negative regulator of PKB/Akt activation by PI 3-K.(Cantley and Neel, Proc Natl Acad Sci USA, 96:4240-5. (1999), Tamura, et al., J Biol Chem, 274:20693-703. (1999)) Loss of PTEN activity results in accumulation of PI(3,4,5)P₃ (Maehama and Dixon, J Biol Chem, 273:13375-8. (1998)), abnormal activation of PKB/Akt and suppression of apoptosis (Haas-Kogan, et al., Curr Biol, 8:1195-8. (1998), Tamura, et al., J Biol Chem, 274:20693-703. (1999)), and increased tumor growth and angiogenesis.(Wen, et al., Proc Natl Acad Sci USA, 98:4622-7. (2001)) These are considered to be the primary mechanisms by which PTEN mutations contribute to unregulated growth, carcinogenesis, and tumor progression.

PTEN was identified as a tumor suppressor that is deleted or mutated in many cancer types.(Li, et al., Science, 275:1943-7. (1997), Teng, et al., Cancer Res, 57:5221-5. (1997)) In one study, PTEN mutations were present in 100% of prostate cancer cell lines, 31 % of glioblastoma cell lines and xenografts, 6% of breast cancer cell lines and xenografts, and 17% of primary glioblastomas.(Li, et al., Science, 275:1943-7. (1997)) Other studies have found PTEN mutations in a significant percentage of primary acute leukemias and non-Hodgkin lymphomas (Dahia, et al., Hum Mol Genet, 8:185-93. (1999)), and in primary and metastatic melanoma.(Birck, et al., J Invest Dermatol, 114:277-80. (2000)) In addition to its association with many cancers, PTEN germline mutations are a hallmark of Cowden's disease, (Liaw, et al., Nat Genet, 16:64-7. (1997), Marsh, et al., Hum Mol Genet, 7:507-15. (1998), Marsh, et al., Genes Chromosomes Cancer, 21:61-9. (1998)) a syndrome which is characterized by benign, hyperplastic, disorganized growths (hamartomas) in multiple organs and an elevated risk of breast and thyroid cancer. Germline mutations in PTEN are also commonly found in individuals with Bannayan-Zonana syndrome (BZS), a similar condition (Marsh, et al., Nat Genet, 16:333-4. (1997)). Assays that accurately determine PTEN activity are very useful in both research and in diagnostics. In addition, if PI 3-K targeted drug therapies are successfully developed for cancer treatment, determination of PTEN status could be a useful predictor of a tumors response to treatment.

A recently characterized lipid phosphatase that may also act in regulation of cell proliferation, and thus in tumorigenesis, is protein tyrosine phosphatase RQ (PTPRQ)(Wright, et al., J. Biol. Chem., 273:23929-23937 (1998), Seifert, et al., Exp Cell Res., 287:374-386. (2003)). Despite its name, the biologically important enzymatic activity of PTPRQ is as a lipid phosphatase. PTPRQ is able to remove phosphates from the 3′, 4′, and 5′ positions of inositol headgroups, although it is most active in removal of 3′ and 5′ phosphates from PI(3,4,5)P₃, PI(3,5)P₂, and PI(4,5)P₂(Oganesian, et al., Proc. Natl. Acad. Sci. USA., 100:7563-7568 (2003)). Overexpression in glioblastoma cells inhibited cell proliferation and caused growth arrest and apoptosis(Oganesian, et al., Proc. Natl. Acad. Sci. USA., 100:7563-7568 (2003)). This effect could result from downregulation of PI 3-K signaling and Akt/PKB activation via dephosphorylation of PI(3,4,5)P₃, similar to the manner in which PTEN acts as a tumor suppressor. Although there is no evidence as of yet for a role for PTPRQ to act as a tumor suppressor, there is likely to be future interest in its potential role in cancer development or progression.

In addition, alterations in SHIP1 activity may be associated with cancers of the blood. Recently, a inactivating mutation in the phosphatase catalytic domain of SHIP1 has been reported in primary myeloid leukemia cells(Luo, et al., Leukemia, 17:1-8. (2003)). SHIP1 is a negative regulator of myeloid cell survival, and loss of SHIP1 activity promotes cell survival and resistance to apoptosis, presumably through deregulation of PI 3-K/Akt signaling (Liu, et al., Genes Dev, 13:786-91. (1999)). Thus, loss of SHIP1 activity may be a factor in the development of acute leukemia and chemotherapy resistance.

PI 3-K activity and cellular levels of PI(3,4,5)P₃ are central to the control of cellular response to insulin stimulation and maintenance of glucose homeostasis.(Okada, et al., J Biol Chem, 269:3568-73. (1994), Czech and Corvera, J Biol Chem, 274:1865-8. (1999)) Insulin stimulation results in tyrosine phosphorylation and activation of the Insulin Receptor Substrate (IRS) tyrosine kinases, which catalyze downstream signaling events which regulate insulin response, including activation of PI 3-K. PI 3-K-dependent signaling is particularly important for regulating several of the metabolic effects of insulin in muscle, such as translocation of the GLUT4 transporter to the plasma membrane (Cheatham, et al., Mol Cell Biol, 14:4902-11. (1994), Hara, et al., Proc Natl Acad Sci USA, 91:7415-9. (1994) Tanti, et al., Endocrinology, 138:2005-10. (1997)), positioning it for glucose uptake. Defects in PI 3-K response to insulin stimulation are associated with non-insulin dependent diabetes mellitus (NIDDM), or “type II” diabetes. In muscle biopsies from NIDDM patients, IRS-1 mediated activation of PI 3-K in response to insulin stimulation is significantly reduced.(Cusi, et al., J Clin Invest, 105:311-20. (2000), Krook, et al., Diabetes, 49:284-92. (2000)) Inhibition of PI 3-K blocks insulin-stimulated glucose uptake and GLUT4 vesicle translocation (Hara, et al., Proc Natl Acad Sci USA, 91:7415-9. (1994)). Mice deficient in PKB/Akt, which is activated by PI 3-K in response to insulin, exhibit a diabetes-like phenotype, including hyperglycemia and defects in glucose uptake in muscle.(Cho, et al., Science, 292:1728-31. (2001)) Altered activity of Akt has also been observed in muscle biopsies from NIDDM patients.(Krook, et al., Diabetes, 47:1281-6. (1998)) PI(3,4,5)P₃ has been pinpointed as the major mediator of the PI 3-K dependent insulin response.(Vollenweider, et al., Mol Cell Biol, 19:1081-91. (1999)). Thus, modulation of PI(3,4,5)P₃ levels by phosphoinositide phosphatase activity is important in the signaling pathways governing insulin-regulated glucose metabolism, and could provide a possible point of intervention for treatment of NIDDM.

There is some evidence that PTEN plays a role in control of insulin sensitivity. PTEN overexpression has been reported to negatively regulate glucose transport.(Nakashima, et al., J Biol Chem, 275:12889-95. (2000)), and the presence of a heterozygous gerinline mutation of the PTEN gene was associated with insulin hypersensitivity and enhanced glucose transport in one clinical study.(Iida, et al., Anticancer Res, 20:1901-4. (2000)) In a mouse model of NIDDM, antisense-mediated ablation of PTEN activity resulted in reversal of hyperglycemia.(McKay, 60th Annual Scientific Sessions of the American Diabetes Association, (2000)) However, more recent studies suggest that endogenous PTEN does not play a role in regulating glucose transport in adipocytes(Mosser, et al., Biochem. Biophys. Res. Commun., 288:1011-1017. (2001), Ono, et al., Mol Endocrinol., 15:1411-1422 (2001)). In addition, because of its importance as a tumor suppressor, targeting PTEN activity would be a difficult strategy for therapeutic intervention.

SHIP2 (SH2-containing inositol phosphatase 2) has recently emerged as a potential therapeutic target for modulating glucose metabolism in NIDDM and insulin resistance. SHIP2 is a second 5′ lipid phosphatase, closely related to SHIP1, that also hydrolyzes PI(3,4,5)P₃ to produce PI(3,4)P₂. (Pesesse, et al., Biochem Biophys Res Commun, 239:697-700. (1997), Ishihara, et al., Biochem Biophys Res Commun, 260:265-72. (1999)) In contrast to SHIP1, SHIP2 is widely expressed in a variety of fibroblast and nonhematopoetic tumor cell lines,(Muraille, et al., Biochem J, 342 Pt 3:697-705. (1999)) and in particular is expressed in target tissues regulating insulin homeostasis.(Ishihara, et al., Biochem Biophys Res Commun, 260:265-72. (1999)) Tyrosine phosphorylation of SHIP2 occurs in response to treatment by a number of growth factors, including insulin, and is thought to act in the regulation of PI 3-K signaling through insulin.(Habib, et al., J Biol Chem, 273:18605-9. (1998))

By modulating the amount of PI(3,4,5)P₃ in insulin-responsive cells, SHIP2 acts as a negative regulator of insulin-induced glucose uptake and glycogen synthesis.(Wada, et al., Mol Cell Biol, 21:1633-46. (2001)) Homozygous deletion of SHIP2 in mice is fatal due to increased insulin sensitivity and severe hypoglycemia, and animals heterozygous for the SHIP2 deletion exhibited increased glucose tolerance and insulin sensitivity, which correlated with increased glycogen synthesis and GLUT4-mediated glucose transport.(Clement, et al., Nature, 409:92-7. (2001)) A recent study reports the presence of SHIP2 gene mutations associated with type 2 diabetes in rats and humans, demonstrating that a deletion identified in the SHIP2 3′ untranslated region of type 2 human diabetic subjects leads to unregulated overexpression of SHIP2 (Marion, et al., Diabetes, 51:2012-7. (2002)). Previous studies have shown that overexpression of SHIP2 inhibits glucose uptake and GLUT4 translocation, producing an insulin-resistant state, an effect dependent on the hydrolysis of PI(3,4,5)P₃ via its 5′ phosphatase activity.(Vollenweider, et al., Mol Cell Biol, 19:1081-91. (1999)) Thus, SHIP2 appears to be an essential negative regulator of insulin signaling and sensitivity, and altered SHIP2 activity may be a key contributing factor to the insulin resistance associated with NIDDM and obesity. Modulation of the lipid phosphatase activity of SHIP2 activity could provide a new approach to therapies for these conditions.

Another 5′ phosphoinositide phosphatase that appears to act in regulation of insulin response is SKIP (Skeletal muscle and kidney enriched inositol phosphatase), which is also highly expressed in insulin responsive tissues(Ijuin, et al., J. Biol. Chem., 275:10870-0875 (2000), Ijuin and Takenawa, Mol. Cell Biol., 23:1209-1220. (2003)). Similar to SHIP2, SKIP appears to play a role in negative regulation of PI 3-K dependent responses to insulin stimulation, in particular glucose transport. SKIP inhibits PI 3-K signaling by conversion of PI(3,4,5)P₃ to PI(3,4)P₂ and is a negative regulator of insulin-induced Akt activation, GLUT4 translocation, and cytoskeletal rearrangement. GLUT4 translocation is substantially inhibited by SKIP overexpression, and this effect is dependent on its 5′ phosphatase activity (Ijuin and Takenawa, Mol. Cell Biol., 23:1209-1220. (2003)). Thus, modulation of SKIP phosphatase activity may prove to be one approach to modulating insulin response in NIDDM.

In summary, the signaling pathways involving these lipid modifying enzymes are often perturbed in the events leading to disease, particularly in non-insulin dependent diabetes mellitus (NIDDM) and cancer. Based on the disclosure contained herein it is evident that the tools developed in the present invention have significant value for research and in diagnostic applications as well as for drug-screening platforms for identification of new lead molecules for therapeutic development.

The most commonly used technique for determining phosphoinositide phosphatase activity is the malachite green assay for the determination of free phosphate generated by enzyme activity. While it is relatively easy to perform, this method has poor sensitivity (200 pmoles phosphate detection limit) and it is prone to interference from organic phosphates that might be present in buffers, cell-culture products, or biological samples. Other approaches for detection of phosphoinositide phosphate activity require enzymatic alteration of radiolabeled or fluorescently-labeled substrates. Detection of phosphatase activity is accomplished by thin layer chromatography to separate substrate and product. This approach is not readily accessible to many researchers, and is clearly not adaptable for high throughput screen(HTS) applications. Thus, there is a need for new reagents and assay formats for detection of phosphoinositide phosphatase activity. Ideally, these assays are non-radioactive, require minimal separation steps, and are specific for the phosphoinositide products of a particular enzymatic reaction. In addition, some assay formats should be homogenous and easily automated for HTS purposes.

Assays that can measure the level of lipid phosphatase activity in tissues have the potential to become powerful research and diagnostic tools. In addition, assay platforms developed for measurement of phosphatase activity in research or clinical settings can be readily modified for use in in vitro assays for novel PTEN, SHIP, and other phosphatase inhibitors. Novel assays for detection of PTEN and SHIP activity disclosed in the present invention are useful for both determination of phosphoinositide phosphatase activity and identification of molecules which regulate the target enzyme activity.

### SUMMARY OF THE INVENTION

It has been recognized that it would be advantageous to develop homogenous HTS-compatible methods for detecting lipid phosphatase activity and the use thereof in disease detection and drug discovery.

The invention provides assays for detecting lipid phosphatase activity using lipid substrates, which may be modified by the incorporation of a fluorescent molecule or other modifications, such as biotinylation. The method comprises the steps of exposing a lipid detector protein containing a lipid recognition motif with a binding specificity for a product lipid of a lipid phosphatase, to a solution containing a substrate lipid of said lipid phosphatase; and determining whether said product lipid is present in said solution.

According to one embodiment of the invention, the lipid phosphatase is SHIP1, SHIP2, or PTEN, and the product lipid is PI(3,4)P₂ or PI(4,5)P₂. In another embodiment of the invention, the lipid phosphatase assay is a screening method for disease detection, i.e. Cowden's disease, and a molecule for treating such disease by detection of alteration of lipid phosphatase activity.

The present invention is further directed to a lipid phosphatase assay kit comprising: a lipid detector protein containing a lipid recognition motif with a binding specificity for a product lipid of a lipid phosphatase, and a solution containing a substrate lipid of said lipid phosphatase. The lipid phosphatase assay kit of the present invention can be a direct assay kit or a competitive assay kit, wherein said product lipid has a stronger affinity to said lipid detector protein than said substrate lipid. The lipid detector protein having a lipid recognition motif may be an antibody against the lipid or a lipid recognition protein(LRP). Various antibodies and lipid receptor proteins that are specific for PI(3,4)P₂ or PI(4,5)P₂ can be used in accordance with the invention. The lipid receptor protein preferably contains an affinity tag fused with the lipid recognition motif. Examples of affinity tags include glutathionine-S-transferase(GST), myc, or FLAG, fused with a lipid recognition motif such as the PH domain. Other affinity tags may also be utilized the only limitation being that of functionality. Additional competing and noncompeting lipids can also be present in the solution, enabling the assay method of the present invention to be used with complex solutions including bodily tissues, fluids, and plasma.

The product lipid is preferably PI(3,4)P₂ or PI(4,5)P₂, which is a de-phosphorylation product of a reaction between the lipid phosphatase SHIP1, SHIP2 or PTEN and the substrate lipid. The lipid phosphatase assay can be used as a screening method for detection of a disease or a drug by detection of a predetermined level of the PI(3,4)P₂ or PI(4,5)P₂ product lipid.

The assay can be any of a number of assay types, but is preferably a plate-based assay. Examples include an enzyme linked immunosorbent assay (ELISA), an amplified luminescence proximity homogenous assay (ALPHA), and a fluorogenic assay such as fluorescence polarization (FP), fluorescence resonance energy transfer (FRET) or time-resolved fluorescence resonance energy transfer (TR-FRET).

An ELISA assay for detection of the products of phosphoinositide phosphatase activity may be designed as either a direct or a competitive assay. In a preferred embodiment where the assay is a competitive ELISA assay, prior to exposing the protein having a lipid recognition motif to a product lipid and a substrate lipid of a lipid phosphatase, a substrate of the assay plate can be coated with the substrate lipid. For example, a biotinylated substrate lipid can be immobilized to a streptavidin-coated substrate. Alternatively, a substrate lipid can be immobilized by covalent attachment of amino-derivitized lipids or by absorption of lipids onto the plate surface. Preferably, the proteins and the lipids of the present invention are not radioactively labeled reagents.

The present invention also provides methods for screening a disease caused alteration of a lipid phosphatase by using the lipid phosphatase assay kit of the present invention to detect changes in the lipid phosphatase activity in bodily tissue, blood, or serum samples. Preferably, the disease is non-insulin dependant, Type II diabetes, Cowden's disease or cancer,

In addition, the present invention provides methods for screening a compound having an enhancing or inhibiting effect on a lipid phosphatase using the lipid phosphatase assay method or the lipid phosphatase assay kit of the present invention to detect changes in the lipid phosphatase activity.

Additional features and advantages of the invention will be apparent from the detailed description which follows, taken in conjunction with the accompanying drawings, which together illustrate, by way of example, features of the invention.

### BRIEF DESCRIPTION OF THE DRAWINGS

The accompanying drawings illustrate the present invention and are a part of the specification. Together with the following description, the drawings demonstrate and explain the principles of the present invention.

FIG. 1 shows a diagram of phosphoinositide interconversions, including the de-phosphorylation of PI(3,4,5)P₃ to PI(3,4)P₂ or PI(4,5)P₂ by SHIP1, SHIP2, or PTEN.

FIG. 2 shows the activity of the lipid phosphatases PTEN and SHIP2 against a variety of substrates, including synthetic phosphoinositides, inositol phosphates, biotinylated phosphoinositides, and fluorescently labeled phosphoinositides.

FIG. 3 illustrates the principles of a competitive ELISA assay for lipid phosphatase activity.

FIG. 4 demonstrates the application of a competitive ELISA assay for the detection of PI(3,4)P₂ produced by SHIP2.

FIG. 5 shows the structures of synthetic phosphoinositides and inositol phosphates and modification via incorporation of fluorescent labels.

FIG. 6 shows the interaction of a PI(3,4)P₂ specific LRP with fluorescently labeled PI(3,4)P₂ in a fluorescence polarization assay.

FIG. 7 shows the specific competition of PI(3,4)P₂ (closed squares) versus PI(3,4,5)P₃ (open squares) with a PI(3,4)P₂ specific LRP for interaction with fluorescently labeled PI(3,4)P₂ in a fluorescence polarization assay.

FIG. 8 demonstrates the application of a competitive fluorescence polarization assay for detection of SHIP2 activity. Formation of product is dependent on reaction time (A), and on enzyme concentration (B).

FIG. 9 shows specific competition of PI(4,5)P₂ versus PI(3,4,5)P₃ with a PI(4,5)P₂ specific LRP in an ALPHA assay.

FIG. 10 shows specific competition of PI(3,4)P₂ versus PI(3,4,5)P₃ with a PI(3,4)P₂ specific antibody in an ALPHA assay.

FIG. 11 illustrates the principles of fluorescence polarization assays for lipid phosphatase activity that incorporate the use of fluorescent substrates.

FIG. 12 illustrates the principles of FRET and TR-FRET assays for lipid phosphatase activity that incorporate the use of fluorescent substrates.

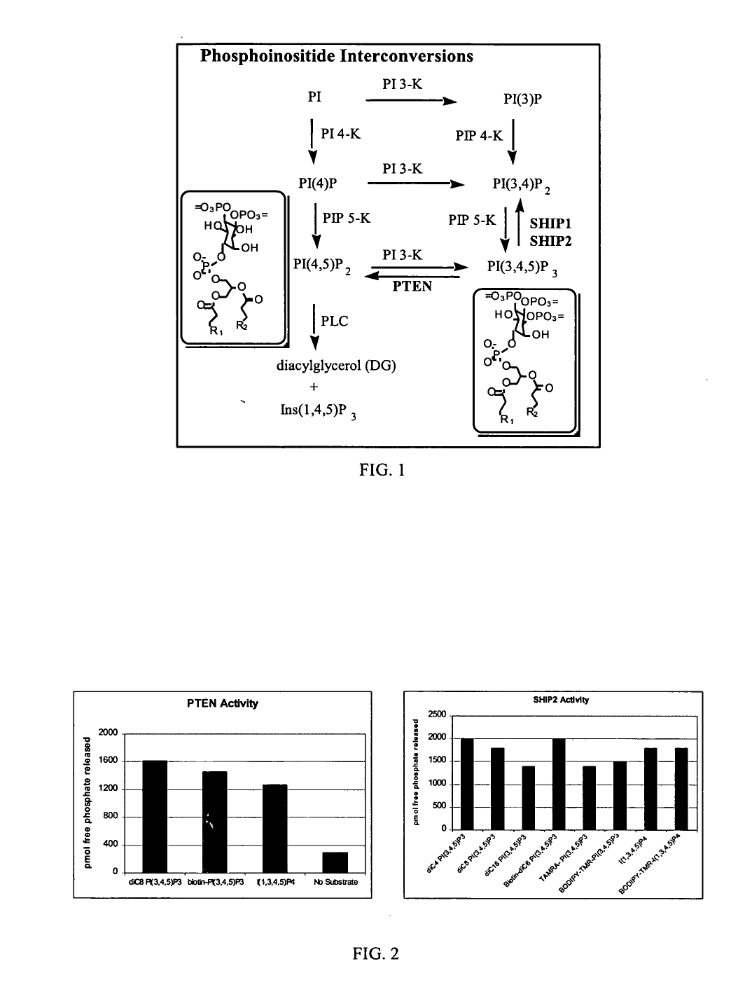

### DETAILED DESCRIPTION

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

The present invention is directed to the use of lipid recognition proteins (LRPs) as detectors of the product lipid of a lipid phosphatase, in a convenient assay platform system that can be readily used in the industry.

The term “lipid phosphatase” refers to an enzyme which changes the phospliorylation state of its substrate, in this case a phosphoinositide lipid or an inositol phosphatase, via removal of a phosphate group. Examples of such lipid phosphatases are SHIP1, SHIP2, PTEN, PTPRQ, SKIP, Myotubularin, MTMR2 and OCRL1.

LRPs that are used in accordance with the present invention can be recombinant proteins expressed as fusions of lipid recognition domains that are present in cellular proteins. The domains of the cellular proteins that interact with a lipid are fused to an affinity tag, such as glutathione-S-transferase (GST), myc, or FLAG, for example. The proteins having such domains are typically involved with such cellular functions as phosphorylation of lipids, or are adaptor proteins that assist in forming complexes with the cellular membrane to allow the cell membrane to interact with lipid structures. The domains from these proteins that allow them to perform such functions can be extracted from the naturally occurring proteins, or prepared through recombinant methods, and can then be fused to GST to form an LRP.

The PH domain is well known to those skilled in the art. Furthermore, different PH domains often exhibit specificity for different phosphoinositides. See Dowler et al., 2000 Biochem J. 351:19-31, which is incorporated herein by reference. All PH domains are predicted to fold into a similar 3-dimensional structure, and may mediate protein-lipid interactions, protein-protein interactions, or both. Polypeptides with PH domains of determined tertiary structure include pleckstrin, spectrin, dynamin, and phospholipase Cγ. Gray et al. ( Anal. Biochem. 313:234-245. (2003)) describe the use of a PH domain derived from the Grp1 protein (General Receptor for Phosphoinositides) in assays designed to detect loss of PI(3,4,5)P₃ substrate as a method for assaying lipid phosphatase activity. The assays described here differ in principle in that they utilize lipid-specific antibodies or LRPs to specifically detect the formation of the product of the enzymatic reaction. Assays by measuring the formation of the reaction products results are more sensitive assays.

In a broad sense, the present invention involves methods, assay kits and apparatuses that use a specific LRP as a detection reagent for specific lipids in an enzyme assay for lipid metabolism. More particularly, the present invention involves the use of a lipid recognition protein(LRP) as a probe that interacts specifically with a product lipid of a lipid phosphatase in such assays. For example, the phospholipase Cδ(PLCδ) and TAPP proteins both include PH domains. While their PH domains vary in the specificity of their interactions with various PIPns, the PLCδ PH domain exhibits a strong preference for interaction with PI(4,5)P₂ and the TAPP PH domains exhibit a strong preference for PI(3,4)P₂. Thus, in a narrow sense, the present invention involves methods that use a lipid detector protein having a binding specificity for a product lipid of a lipid phosphatase as a lipid detection probe to measure the product lipid of a lipid phosphatase. Therefore, minimal components of a phosphoinositide phosphatase assay of the present invention are 1) a source of enzyme, 2) the appropriate phosphoinositide substrate, and 3) a detection reagent (either a LRP or antibody) with specificity. for the phosphoinositide product of the enzymatic reaction.

For assays of phosphatase activity in cells or tissues, PTEN or SHIP can be isolated by immunoprecipitation using specific antibodies and protein A-agarose. Antibodies suitable for isolation of PTEN or SHIP are available from several commercial sources, including Upstate Biotechnology (NY) and Santa Cruz Biotechnology (CA). For the purposes of assay application to HTS and drug discovery, a source of recombinant enzyme is preferable, i.e., vectors for recombinant expression of affinity tagged PTEN in bacteria. Other sources of phosphoinositide phosphatase expression systems are also suitable.

A number of options are available for obtaining enzyme substrate mixtures. Both PTEN and SHIP use PI(3,4,5)P₃ as their preferred substrate. Synthetic short-chain PI(3,4,5)P₃ (diC₄, diC₈) has the advantage of being soluble in aqueous solution without requiring incorporation into liposomes or micelles. The substrate may also be present in the form of PI(3,4,5)P₃ incorporated into liposomes or micelles. This requires the mixing of long acyl-chain (diC₁₆ to diC₂₄) PI(3,4,5)P₃ with the appropriate ratios of carrier lipids, such as phosphatidylcholine or phosphatidylethanolamine, in organic solvent. Following evaporation of the solvent to produce a lipid film, water or a suitable buffer solution is added to rehydrate the lipids. Micelles may be prepared by sonication of the mixture, while passage through an extruder is necessary to produce a homogenous population of bilaminar liposomes.

Soluble diC₄ or diC₈ PI(3,4,5)P₃ are the straightfonvard choice as substrate in an in vitro assay. It has been shown that the diC₄ and diC₈ synthetic phosphoinositides arc suitable as a substrate. In addition, a fluorescently-labeled diC₆ PI(3,4,5)P₃ has already been shown to act as a substrate for PTEN. FIG. 2 shows the activity of SHIP2 and PTEN assays against selected phosphoinositide and inositol phosphate substrates by determination of free phosphate using malachite green (Biomol, Pa.). While it is necessary to test each enzyme to determine which method of substrate presentation is optimal, these results demonstrate that soluble diC₄ and diC₈ phosphoinositides are suitable substrates for use in lipid phosphatase assays.

One embodiment of the present invention is a Enzyme Linked Immunosorbent Assay (ELISA) of a lipid phosphatase. In order to readily distinguish PI(4,5)P₂ from other PIPs, a derivative such as biotinylated diC₆ PI(4,5)P₂ is immobilized in the wells of streptavidin-coated assay plates. As an example, a 96-well plate can be used, although the present embodiment of the invention is clearly adaptable for a variety of assay plate sizes and formats. Initial experiments establish the range of LRP detector protein and biotinylated diC₆, PI(4,5)P₂ concentration where detection of the target phosphoinositide is optimized in an assay tray format. For example, in a standard curve binding procedure, a 96-well streptavidin-coated assay plate marketed as StreptaWell® (Roche) is coated with increasing amounts of biotinylated PI(4,5)P₂ per well. The coated wells are then blocked for an hour at room temperature using 100 μL of Stabilguard® (SurMedics) per well. The samples are then incubated with 10 pmol of GST-tagged LRP in a 100 μL volume per well. Several washes are then performed. Next, 100 μL of a 1:1000 dilution of an anti-G ST HRP-conjugated antibody, provided by Sigma, is added to each well. The GST HRP-conjugated antibody is provided as a reagent that interacts with the GST portion of the lipid recognition protein, and hence allows for subsequent calorimetric detection. After one hour of incubation at room temperature, the plates are washed and 100 μL of 3′,3′,5′,5′-tetramethylbenzidine (TMB) substrate solution as a development reagent (Sigma) is added to each well. Following color development, the reaction is stopped by the addition of 100 μL 0.5 M H₂SO₄ and the absorbance at 450 nm is measured. The results of the absorbance measurements represent the binding of the LRP detector protein to increasing amounts of immobilized diC₆ PI(4,5)P₂. A competition procedure can be performed using similar methodology. Assay plates such as the StreptaWell® micro plates, used in the previously discussed optimization process, are prepared by coating the wells with 10 pmol of biotinylated diC₆ PI(4,5)P₂ per well. In a separate incubation apparatus, 10 pmol of LRP is preincubated with increasing amounts of closely related derivative PIPs. For example, the LRP is preincubated with either diC₈ PI(4,5)P₂ or diC₈ PI(3,4,5)P₃, prior to binding to the biotinylated diC₆ PI(4,5)P₂ coated surface of the assay plate wells.

The results of the competition procedure can be determined by, for example, measuring absorbance (450 nm) for each of PI(4,5)P₂ and PI(3,4,5)P₃ at various pmol increments of competing PIP. The difference in competitiveness is particularly evident at lower levels of PIP, and the difference will clearly exemplify the ease with which the assay can distinguish between the two phosphoinositides.

In the application of the ELISA format for detection of a lipid phosphatase activity, recombinant phosphatase enzyme is incubated with substrate for one hour. Enzyme activity is stopped and the reaction mixtures are pre-incubated with LRP and then tested in a competition binding assay. A standard curve in which increasing amounts of competitor is added to the assay is run alongside the enzyme reactions. The degree of PI(3,4,5)P₃ conversion to PI(3,4)P₂ or PI(4,5)P₂ is then estimated by comparing the values obtained for the reaction mixtures in the competition ELISA. An ELISA-based lipid phosphatase assay kit is of use to individual researchers, and overcomes the shortcomings of radioactive labeling and separation methods which are used in the related art.

Most experiments testing for lipid phosphatase activity exemplify actual cellular conditions in vivo where the level of substrate conversion is approximately 5 to 15%. Because the ability of the LRP detector protein to distinguish between differences in the amount of the product lipid that is present appears to be more sensitive when it is present at less than 50% of the lipid mixture, the assay of the present invention is well suited for detecting differences in the range for which tests are commonly performed.

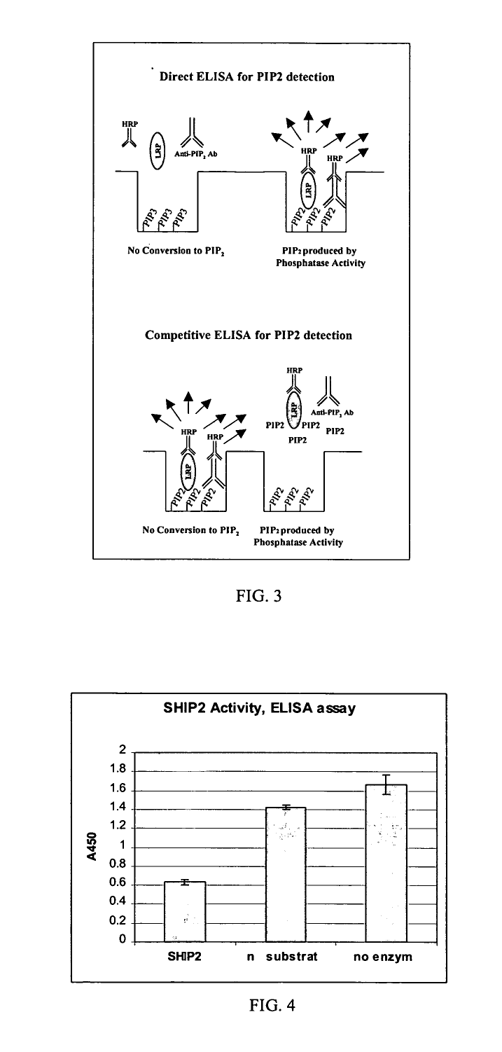

An ELISA assay for detection of the products of phosphoinositide phosphatase activity may be designed as either a direct or a competitive assay, as illustrated in FIG. 3. PI(3,4,5)P₃ substrate is incubated with either PTEN or SHIP enzymes for conversion to PI(4,5)P₂ or PI(3,4)P₂ respectively. In a direct assay, PI(3,4,5)P₃ substrate may be immobilized directly in an assay plate. For substrate conversion, the enzyme solution may be added directly to the plate, and then removed to stop the reaction. The appropriate LRP or antibody reagent is then added to the plate, followed by several washes, then addition of the appropriate HRP-conjugated secondary antibody. Conversion of PI(3,4,5)P₃ to the relevant diphosphate form is determined by the extent of LRP or antibody binding, as measured by HRP activity. For colorimetric detection TMB substrate solution (Sigma) is added, followed by absorbance measurement at 450nm after the addition of 0.5 M H₂SO₄ to stop color development. Chemiluminescent detection is also an option using HRP or Alkaline Phosphatase luminescent substrates (SuperSignal® from Pierce for example).

Biotinylated diC₆ PI(3,4,5)P₃ (Echelon) may be bound to the surface of a streptavidin-coated assay plate to be used directly as the lipid substrate. One prerequisite of this assay method is that the biotinylated form of PI(3,4,5)P₃ acts as a suitable substrate for PTEN or SHIP when immobilized in the wells of the microtiter plate. If for some reason the enzyme prefers that the biotinylated PI(3,4,5)P₃ be free in solution rather than immobilized on a plate, the enzymatic reaction may be performed, followed by transfer of the phosphoinositide mixture to the plate to allow binding of the biotinylated lipids in the mixture prior to detection. The ability of the enzyme to use biotinylated lipids as a substrate may be tested by commonly used methods, such as determination of free phosphate by malachite green or by thin layer chromatography.

Another option for development of an ELISA is a competitive assay in which the products of the enzymatic reaction compete for antibody or LRP binding to a PI(3,4)P₂ or PI(4,5)P₂ coated microtiter plate. In this case, PI(3,4,5)P₃ substrate is incubated with the enzyme, and the reaction mixture is preincubated with an LRP or antibody detection reagent, and is then added to a detection plate coated with PI(3,4)P₂ or PI(4,5)P₂. The presence of PI(3,4)P₂ or PI(4,5)P₂ in the competing mixture blocks the LRP or antibody binding and results in reduced signal. We implemented a competitive ELISA for the detection of SHIP2 activity. In this case the enzyme product, PI(3,4)P₂, competes for binding of the GST-TAPP detector protein to a PI(3,4)P₂ coated plate. As the products of the enzymatic reaction increase, binding of the detector protein to the plate decreases, giving a decrease in absorbance.

The assay involves four steps. First, the SHIP2 enzyme and diC₈ PI(3,4)P₂ substrate were combined in an appropriate reaction buffer and the reaction allowed to proceed. A solution of GST-TAPP1 was added to the reaction mixture, and pre-incubated for an hour. Following the preincubation step, the mixture was transferred to the wells of a PI(3,4)P₂-coated microplate for binding. Binding was detected using a HRP-conjugated anti-GST antibody, followed by colorimetric development and absorbance measurement. FIG. 4 shows the application of this assay for the detection of SHIP2 activity. Substrate conversion can be quantified by comparison to a standard curve of PI(3,4)P₂. This assay can be applied to detection the activity of other 5′ phosphosinositide or inositol phosphate phosphatases, including SHIP1 and SKIP.

In fluorescence polarization assays, light from a monochromatic source passes through a vertical polarizing filter to excite fluorescent molecules in a sample tube or microplate well. Only those molecules that are oriented in the vertically polarized plane absorb light, become excited, and subsequently emit light. The emission light intensity is measured both parallel and perpendicular to the excitation light. The fraction of the original, incident, vertical light intensity that is emitted in the horizontal plane indicates the amount of rotation that the fluorescently labeled molecule has undergone while in the excited state, and is a measure of its relative size. Changes in the relative size of a fluorescently labeled molecule may be due to interactions with another molecule, dissociation, enzymatic modification, degradation, or conformational change.

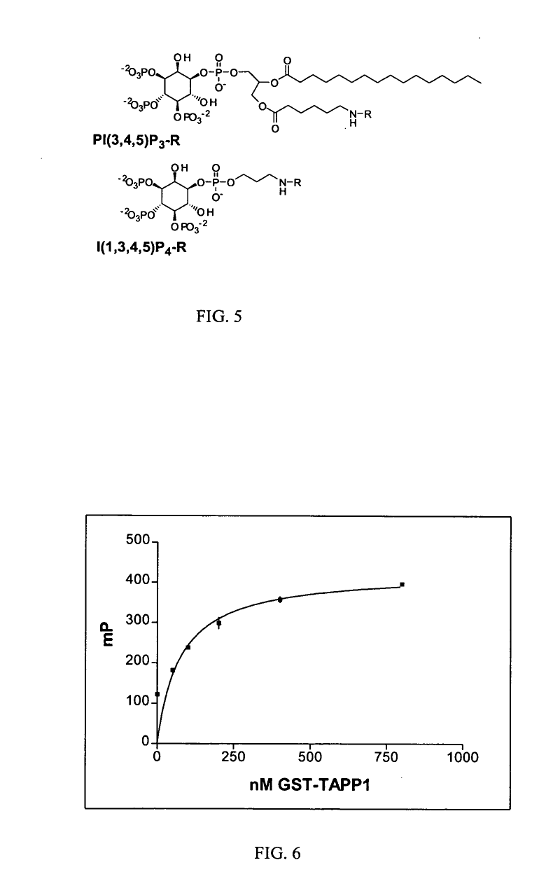

Synthetic PI(3,4)P₂ and PI(4,5)P₂ may be conjugated to several types of fluorescent molecules via modification of the sn-1-O-acyl chain fatty acid (FIG. 5). These can be used in the development of fluorescence polarization assays for lipid phosphatase activity. When excited with plane polarized light, an isotropically, rapidly tumbling fluorescent phosphoinositide probe emits light with no net polarization. When complexed to the antibody or LRP, the tumbling rate slows and under these anisotropic conditions, the emission retains polarization in direct proportion to the fraction of probe bound. Light emitted by the lipid/protein complex will be more polarized, and the fluorescence polarization value (mP) of the sample will be higher. In a competition assay with free PI(3,4)P₂ or PI(4,5)P₂ produced by the activity of SHIP or PTEN, mP values should decrease as the unlabeled product of the enzymatic reaction competes with the fluorescent lipid probe for interaction with the LRP or antibody.

In a competitive fluorescence polarization assay for SHIP2 activity, potential PI(3,4)P₂ detection reagents include antibodies and recombinant LRP, GST-TAPP1 is preferred for use as a detector for PI(3,4)P₂. The interaction of recombinant GST-TAPP1 with BODIPY®-FL and BODIPY®-TMR labeled PI(3,4)P₂ tracers was tested. Tracer concentrations were held at 10 nM and increasing amounts of protein were added to determine the concentration required for maximal capture. Greater maximal binding and affinity was observed for the interaction of the GST-TAPP1 with BODIPY®-TMR labeled tracer when compared to BODIPY®-FL. The change in polarization values produced by protein interaction with the red fluorophore labeled tracer was also considerably higher. Bₘₐₓ and K_{d} values for binding of GST-TAPP1 to a BODIPY®-TMR -PI(3,4)P₂ tracer at 5 nM were 425 mP and 75 nM respectively (FIG. 6).

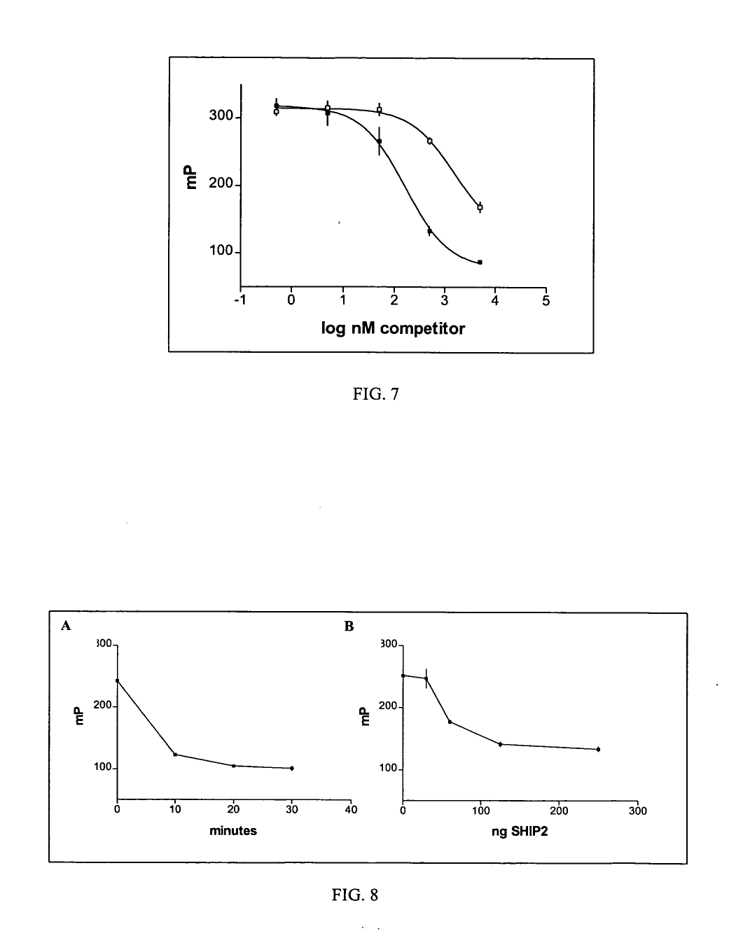

In order to use these reagents inma competitive FP assay, unlabeled PIP products of an enzymatic reaction must successfully compete with the labeled tracer for interaction with the binding proteins. In addition, the level of cross-reactivity of binding proteins with the PIPs used as enzyme substrates should be minimal. Competition assays were performed to examine the selectivity and sensitivity of PI(3,4)P₂ detection by GST-TAPP1 (FIG. 7). Binding to a BODIPY®-TMR-PI(3,4)P₂ tracer was displaced by diC₈-PI(3,4)P₂ with an IC₅₀ value of 165 nM and by diC₈-PI(3,4,5)P₃ with a predicted IC₅₀ of 1508 nM. This degree of selectivity was judged to be sufficient for designing a SHIP2 assay, as long as the initial amount of PI(3,4,5)P₃ substrate was kept low enough to minimize competition.

The assay has four steps. First, SHIP2 enzyme and substrate are combined in an appropriate reaction buffer and the reaction is allowed to proceed. Following the incubation period, a solution of GST-TAPP1 was added and mixed, followed by addition of a fluorophore-labeled PI(3,4)P₂ tracer. Finally, polarization values were measured to determine the extent of enzyme activity. All reaction and detection steps can be performed directly in microplate wells.

Optimized assays for the detection of SHIP2 activity were evaluated as follows. Reactions were performed in 100 mM Tris, pH 7.4, 150 mM NaCl, and 5 mM MgCl₂, using diC₈-PI(3,4,5)P₃ as the substrate. Time dependence of substrate conversion was demonstrated using 125 ng of enzyme and 2.5 μM of substrate per reaction. For enzyme titration experiments, substrate concentration was held constant at 2.5 μM while increasing amounts of SHIP2 were added. Reactions were allowed to proceed for 30 minutes at 25° C. GST-TAPP1 was added to give a final concentration of 100 nM and BODIPY®-TMR-PI(3,4)P₂ was added to give a final concentration of 2.5 nM. Final sample volumes were 50 μL. The production of PI(3,4)P₂ was detected as a decrease in mP values as enzyme products compete with PI(3,4)P₂-BODIPY®-TMR for interaction with the binding protein. PI(3,4)P₂ production was dependent on reaction time (FIG. 8A) and on enzyme concentration (FIG. 8B). Characteristics of this assay showed suitability for use in HTS applications, including a Z′ value greater than 0.5, lack of interference by DMSO and MP value stability over time. This assay can be applied for detecting the activity of other 5′ phosphoinositide or inositol phosphate phosphatases, including SHIP1 and SKIP.

One advantage of these “mix and measure” FP assays is the lack of requirement for additional assay components. FP approaches also require that only the lipid component of the assay be labeled with a fluorescent tag, in contrast to FRET and homogeneous time-resolved fluorescence (HTRF) methods, which require multiple labeling reactions(Pope, et al., Drug Discov Today, 4:350-362. (1999)). FP measurements are ratiometric rather than intensity-based, giving several advantages including greater ease of miniaturization, over other fluorescent assay approaches in which values are based on signal intensity.(Pope, et al., Drug Discov Today, 4:350-362. (1999, Kowski and Wu, Comb Chem High Throughput Screen, 3:437-44. (2000)) and less susceptibility to artifacts produced by exogenous fluorescence or quenching. The probability of these types of interference is even further reduced by the use of red-shifted tracers.

FP applications have been used successfully in many types of assays, including competitive immunoassays to detect drugs and small molecules, and assays for detection of enzyme activity. Fluorescence polarization has multiple advantages compared to other assay methods, in particular, there is no need for secondary detection reagents, it is a homogenous technology, reactions are very rapid, and it is readily adapted for automation.

The principles of the present invention can also be applied to other assay methods, such as the type using ALPHAScreen® reagents and the Fusion Alpha Universal Microplate Analyzer from PerkinElmer Life Sciences. The ALPHAScreen system detects emission shifts due to reactions involving the transfer of singlet oxygen. More particularly, the system uses photosensitive donor beads which convert ambient oxygen to a singlet state upon illumination at 680 nm. If an acceptor bead is in close proximity to the donor bead, due to a biological interaction, the diffusion of singlet oxygen activates chemiluminscent receptors and fluorescent acceptor molecules on the bead, resulting in an emission shift from 520 to 620 nm.

The ALPHA assay format is an example of yet another assay method that can be used in accordance with the principles of the present invention. The ALPHA format has the advantages of high specificity and sensitivity, and requires significantly less protein and lipid reagents than the ELISA assay format.

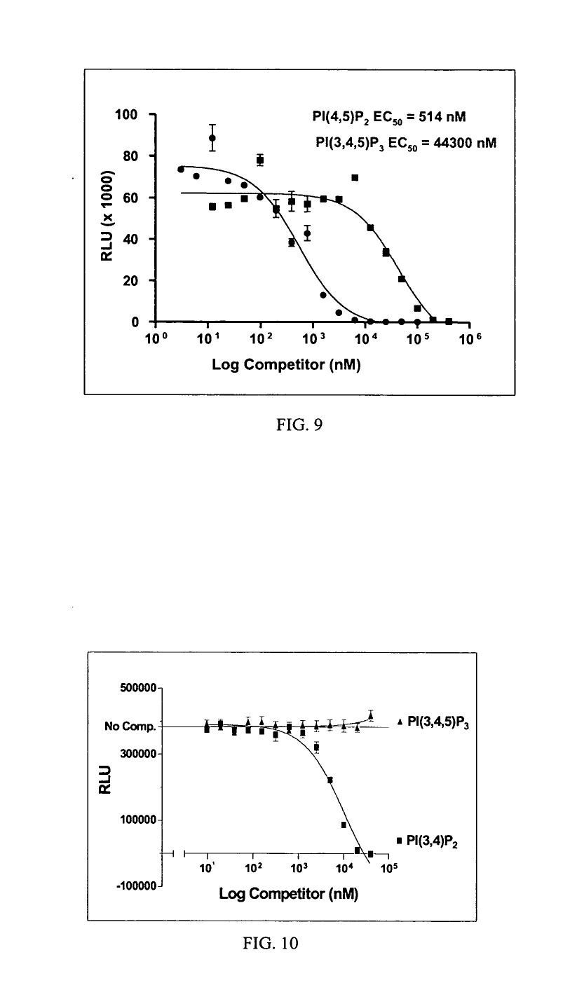

The present invention provides ALPHA assays to detect specific binding and competition of phosphoinositide diphosphates using antibodies or LRPs. This approach can be used in competitive assays for the detection of PI(3,4)P₂ and PI(4,5)P₂ produced by lipid phosphatase activity. FIG. 9 shows the results of a binding and competition experiment using a PI(4,5)P₂-specific LRP derived from PLCδ and biotinylated PI(4,5)P₂. Increasing amounts of unlabeled PI(4,5)P₂ or PI(3,4,5)P₃ were added. PI(4,5)P₂ competition is approximately 90-fold greater than PI(3,4,5)P₃ competition, indicating a high degree of specificity for recognition of PI(4,5)P₂. FIG. 10 shows a similar competition experiment using anti-PI(3,4)P₂ monoclonal antibodies, described previously. The antibody shows a high degree of specificity for PI(3,4)P₂ versus PI(3,4,5)P₃.

The ALPHA assay may be implemented as a competitive assay for the detection of PTEN or SHIP activity. PI(3,4,5)P₃ substrate will be incubated with either PTEN or SHIP, and the resulting lipid mixture will be added to ALPHAScreen donor and acceptor beads. For example, for detection of PI(4,5)P₂ production by PTEN, components of the reaction mixture will be streptavidin-coated donor beads, biotinylated PI(4,5)P₂, a PI(4,5)P₂-specific GST-tagged LRP or IgG antibody and either anti-IgG or anti-GST acceptor beads. An assay designed to detect PI(3,4)P₂ produced by SHIP would contain protein detection reagents or antibodies with specificity for that phosphoinositide product.

Furthermore, the principles of the present invention can be readily applied to other assay formats for detection of lipids or associated enzyme activity using the LRPs or lipid-specific antibodies as detection devices. This includes homogeneous fluorescence methodology, in particular FP, FRET, and TR-FRET. In “direct” fluorescence-based assays, enzyme alteration of a fluorophore-labeled lipid substrate produces a change in interaction with a LRP or antibody. A similar type of direct assay for detection of protein kinase activity using fluorescent substrates has been developed and marketed by Molecular Devices (Sunnyvale, Calif.) (Gaudet, et al., J. Biomol. Screening, 8:164-175. (2003), Sportsman, et al., Comb. Chem. High Throughput Screening, 6:195-200. (2003)). In addition to competitive assays, it may also be advantageous to develop assays in which enzymes act directly upon a fluorescently labeled lipid substrate to produce a change in its interaction with a detector protein or antibody.

Development of these assays requires that lipid phosphatases be able to metabolize fluorophore-labeled PIP substrates. Fluorophore-labeled diC₆ PIPs and P-1 aminopropyl-modified IPs can be synthesized. For example, dyes can be covalently attached to the end of one of the acyl chains for the PIPs and at the P1 phosphate for the IPs, and are readily incorporated into synthetic PIPs and IPs as the final synthetic steps. Sample structures of fluorophore labeled PIP and IP tracers are shown for PI(3,4,5)P₃ and I(1,3,4,5) P₄ (FIG. 5). A wide variety of dyes can be used as labels in FP assays without compromising interactions with specific protein detectors. It has been shown that SHIP2 has activity against fluorophore-labeled PIP substrates, and published work on PTEN indicates that it has similar activity(Maehama, et al., Anal Biochem, 279:248-50. (2000)). Further studies of enzyme activity toward and preference for different fluorescent substrates can be easily done using the malachite green assay for phosphate release. A second requirement is that the detector proteins used must show sufficient specificity for the detection of the fluorescent PIP product versus the fluorescent PIP substrate. For PTEN and SHIP assays, PLCδ and TAPP1 are preferred LRPs respectively.

Other major variables to address are the concentration of fluorescent substrates in the enzymatic reaction, the final concentration of fluorescent PIPs in the assay read step, and the amount of detector protein required for optimal signal and sensitivity. Other than optimal substrate concentration, these variables will need to be addressed separately for FP and FRET assays. Approach used for FP assays can be modified appropriately for FRET assays.

In competitive FP assays, where the unlabeled products of the enzyme reaction compete with a fluorescent tracer for interaction with the detector protein, assay performance is best when the concentration of fluorescent PIP tracer is in the 5-20 nM range, and it is necessary to keep tracer concentrations low to maximize sensitivity. However, in assays that use fluorescent substrates directly, higher concentrations, in the 20 to 100 nM range, can be tolerated in the final read step (Sportsman, et al., Comb. Chem. High Throughput Screening, 6:195-200. (2003)). The increased amount of fluorescence is actually advantageous, as it decreases standard deviation in fluorescence polarization measurements (Owicki, J. Biomol. Screening, 5:297-306 (2000)) and increases tolerance to spectral interference when screening compounds. The optimal final concentration of fluorescent PIP and the optimal amount of detector protein can be determined by modeling conversion of fluorescent PI(3,4,5)P₃ to either PI(3,4)P₂ or PI(4,5)P₂ using mixtures containing different ratios of the substrate and product lipids. Concentrations of 10 to 100 nM total lipid will be tested with varying concentrations of detector protein, from 10 to 500 riM. In competitive FP assays, the optimum ratio of detector protein to lipid tracer varies from 1:1 to 1:5, depending on the specific protein-lipid pair. Ideally conditions will allow detection of 10% or less substrate conversion and a change in mP values of 200 or greater for an FP assay.

With competitive assays, it is advantageous to keep the substrate concentration at or near the Kₘ value of the enzyme, usually in the 2-5 μM range. The optimal concentration of fluorescent substrate to use in the enzyme reaction step can be determined by setting up reactions containing a constant amount of enzyme with increasing amounts of substrate. Substrate concentrations are preferably at or below the Kₘ value of the enzyme (approximately 5 μM for SHIP2), so that the amount of product will vary linearly with time. After reactions are complete, aliquots of each reaction will be diluted into buffer containing the detector protein, so that the final concentration of fluorescent PIP is equal for all samples to be read. For example, if the desired final concentration of fluorescent PIP is 20 nM, reactions containing 1 μM of substrate will be diluted 1/50, while reactions containing 500 nM substrate would be diluted 1/25. This will determine the Kₘ values and substrate dependence of enzyme activity for fluorescent PIP substrates using the optimal conditions for total amount of fluorescent PIP and detector protein determined previously.

Since the optimal substrate concentration in the enzymatic reaction and the optimum amount of PIP in the final step may differ by 10-fold or more, it will most likely be necessary to incorporate a dilution step into the final assay protocol. This is true of other FP assays that use fluorescent substrates for detection of protein kinase activity (Gaudet, et al., J. Biomol. Screening, 8:164-175. (2003, Sportsman, et al., Comb. Chem. High Throughput Screening, 6:195-200. (2003)). Ideally, for HTS purposes, it would be most advantageous if both reaction and dilution/detection steps could be performed in a single well of an assay plate. For example, 5 μl enzyme reactions might be performed using fluorescent PI(3,4,5)P₃ substrate, and detection of enzymatic activity would be performed via direct addition of a mixture containing the detector protein and the appropriate quencher, to give a final volume of 25 or 50 μl. However, if a greater degree of dilution is required, part of the enzyme reaction could be transferred to a separate well containing the detector protein mixture.

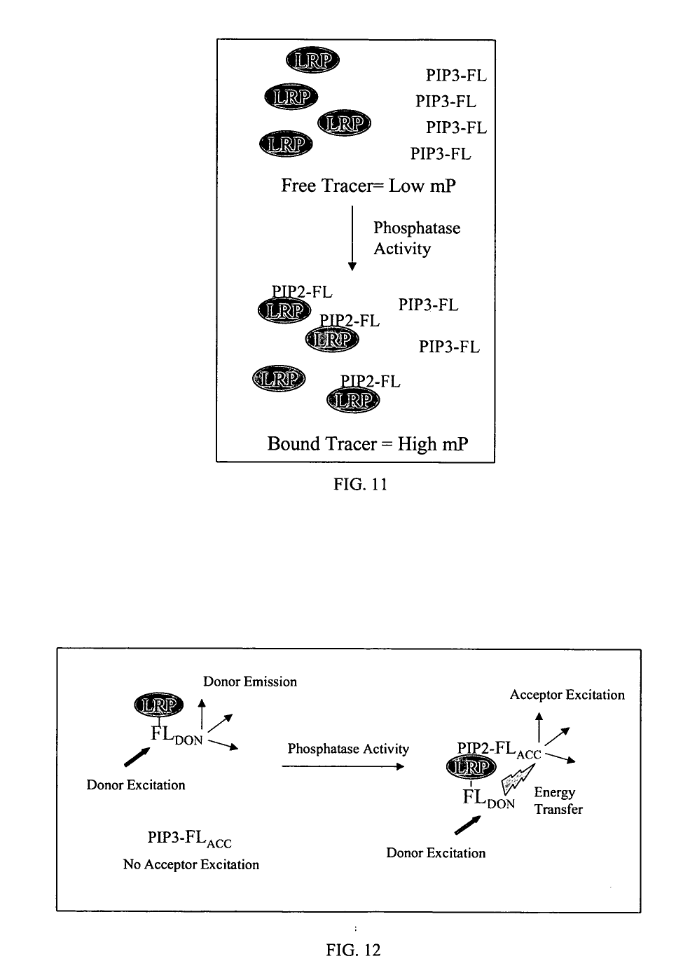

The present invention also provides fluorescence polarization(FP) assays of a lipid phosphatase using fluorescent substrates. In this type of assay, the fluorescent products of the phosphatase reaction will interact with the detector to produce an increase in polarization values (mP) that is proportional to enzyme activity, as diagrammed in FIG. 11.

Preferably, PLCδ is used as the LRP detector for PI(4,5)P₂ produced by PTEN activity, and TAPP1 is used as the detector for PI(3,4)P₂ produced by SHIP activity. Other LRPs or antibody detectors may also be used in the present invention. It is shown in the present invention that red-shifted dyes, such as Tetramethylrhodamine and BODIPY®-TMR are suitable labels for the PIP tracers in competitive FP assays. Once specificity of the protein-tracer interaction has been demonstrated, the conditions best suited for detection of enzyme activity, including substrate concentrations, can be determined.

Fluorescence resonance energy transfer (FRET) is a distance-dependent interaction between the electronically excited states of two dye molecules. The donor emits excited-state energy at a wavelength within the excitation spectrum of the acceptor, this energy transfer results in the emission of fluorescence from the acceptor. The emission spectra of the donor and the excitation spectra of the acceptor must have sufficient overlap, as diagrammed on the right, for this to be efficient. The distance between the donor and acceptor molecules is also important, as energy transfer is most efficient over distances of less than 100 Å. FRET has been applied to investigating a variety of biological phenomena that produce changes in molecular proximity.

Time-resolved fluorescence energy transfer (TR-FRET) is a variation of FRET that makes use of long-lived fluorescent molecules as donors. Chelates of rare earth elements (lanthanide chelates) are used as donors, with conventional fluorescent molecules as acceptors. The advantage of using the lanthanide chelates as donors is that their excited-state lifetimes are on the millisecond time scale, while that of most small-molecule fluorophores is on the nanosecond time scale. Thus, a delay of 100 μs before measuring the fluorescence of either the donor or the acceptor species can be used to “gate out” interfering fluorescence arising from matrix components, library compounds or laboratory plastics. This gated detection method also reduces fluorescence signals arising from direct excitation of the acceptor fluorophore. The signal-to-background ratio for TR-FRET is typically several fold higher than is generally seen with standard, shorter lifetime FRET pairs. Additionally, the large Stokes shift of the lanthanide chelates (>200 nm) helps to decrease background fluorescence levels. Table 2 shows some of the donor and acceptor pairs that can be used for FRET and TR-FRET. Most of these are commercially available in formulations suitable for conjugation to proteins or our synthetic lipids. Due to its advantages over conventional FRET, the present invention provides reagents and protocols for use in TR-FRET assays for phosphatase activity.

TABLE 2
Possible Donor and Acceptor Pairs for
use in FRET and TR-FRET assays

| | |
|---|---|
| Donors | Acceptors |

FRET

| | |
|---|---|
| Fluorescein | Tetramethylrhodamine |
| Tetramethylrhodamine | Texas Red |

TR-FRET

| | |
|---|---|
| Europium (Eu) | Cy5 ® |
| | Allophycocyanin (APC) |
| | AlexaFluor ® 647 |
| Terbium (Tb) | Fluorescein |
| | Rhodamine |
| | BODIPY ®-TMR |
| | BODIPY ®-FL |

TR-FRET assays can be implemented as either competitive or direct assays. In both cases, the assay detects interaction of a lanthanide-derivative labeled antibody or LRP with an acceptor fluorophore-labeled PIP. In a competitive TR-FRET, the PH domain of an LRP, which specifically binds to a product lipid of a lipid phosphatase, i.e. P(4,5)P₂, can be directly labeled with an Eu chelate. An appropriate PIP conjugated to Cy5® or AlexaFluor® 647 can be used as the binding partner.

Although the present invention can be implemented as a competitive assay, TR-FRET assays are preferred to detect conversion of a fluorophore-labeled PIP substrate. FIG. 12 shows a schematic of such an assay. LRPs will be labeled with an Eu chelate, as the donor molecule, according to the manufacturer's protocols (Amersham, or PerkinElmer Life Sciences). AlexaFluor®647-labeled PIPs can be used as the acceptor. Alternatively, Biotinylated PIPs complexed to APC-streptavidin are also suitable. Binding curves can be performed to examine the specificity of the interactions of the labeled LRPs, for example, to verify that Eu-TAPP1 interacts specifically with AlexaFluor®647-PI(3,4)P₂ and not AlexaFluor®647-PI(3,4,5)P₃. Because labeling the LRP with Eu could produce conformational changes, it may be necessary to adjust labeling conditions to avoid compromising the specificity of the LRP-PIP interaction. Once this specificity of the protein-tracer interaction has been demonstrated, the conditions best suited for detection of enzyme activity can be determined in experiments that model substrate conversion under different conditions. In addition, since the activity of lipid phosphatases toward AlexaFluor® and Cy5® labeled PIPs and IPs has not been demonstrated, the activity of PTEN and SHIP2 enzymes toward these substrates can also be tested.

The present invention also provides a kit or assay for determining PTEN activity in cells and tissues. Given the importance of PTEN in cancer development and progression, the assays provided by the present invention would be very useful to cancer researchers in both basic science and clinical areas.

In a competitive ELISA for PTEN activity, the lipid products of the enzymatic reaction will compete for antibody or LRP binding to a PI(4,5)P₂ coated microtiter plate. In this case, PI(3,4,5)P₃ substrates are incubated with enzyme, and the reaction mixture preincubated with the LRP or antibody detection reagent, is then added to a detection plate coated with PI(4,5)P₂. The presence of PI(4,5)P₂ in the competing mixture should block the LRP binding and result in a reduced signal. Standard protocols for binding biotinylated phosphoinositides to streptavidin-coated plates, the blocking, incubation, and washing steps have already been established and should be readily adapted and applied for use in detection of PTEN activity.

A competitive ELISA is preferred in a typical usage scenario for detection of cellular PTEN activity. PTEN would be isolated by immunoprecipitation from cultured cells. Immunoprecipitation can be performed using anti-PTEN antibodies from commercial sources, and the immuno-complex would be bound to protein A beads, according to usual protocols. Enzyme-bound beads would then be incubated with either PI(3,4, 5)P₃ or I(1,3,4,5)P₄ substrate for the appropriate time. The reaction would be stopped by removing the mixture from the beads. Detection of PI(4,5)P₂ would be accomplished by incubation with an appropriate LRP, either PLCδ or a newly engineered LRP, followed by addition to wells of a PI(4,5)P₂ coated plate. The extent to which the LRP or binding to the plate is decreased by competition should reveal the extent of substrate conversion when compared to a standard curve of increasing PI(4,5)P₂, which will be included with the assay.

The lipid detection reagent for developing these assays is preferably PLCδ, although other antibodies and LRPs are also suitable. The optimum amount of LRP and immobilized PIP, and the sensitivity and specificity of competition by PI(4,5)P₂ and other PI(3,4,5)P₃s can be determined. It was found that decreasing the amount of PIP bound to the plate results in a more sensitive assay without decreasing signal intensity. Other parameters to optimize include incubation times, buffer composition, and choice of a suitable secondary detector. The ELISA assay may also be used to detect changes in PTEN activity between normal and PTEN mutant cell lines. This assay may be applied for the determination of PTEN levels in tissues, including tumor samples. In addition, the present invention also provides screening methods for detection of a disease caused by alteration of a lipid phosphatase, or compounds which inhibit or enhance the activity of a lipid phosphatase.

In addition to the above-described advantages of the assay methods of the present invention, a non-radioactive assay using an LRP as a lipid detection reagent for assaying enzyme activity readily lends itself to automation. Assay platforms used in the nonradioactive assay can be used, for example, with an automatic analyzer such as the Fusion Universal Microplate Analyzer by Perkin Elmer Life Sciences. Such a device is easily integrated with automated systems for plate stacking, liquid handling and cell-based assays. Other automation can be applied to the process in order to increase the assay process rate, including a plate washer, harvester and plate scintillation counter, such as Orca/Biomek®.

The determination of a lipid phosphatase activity in biological samples can also be determined using the plate-based assays described above. The principles of the present invention may be applied in a clinical assay setting. A clinical assay can be performed for phosphatase activity that is suitable for analysis of small, less invasive clinical samples, such as blood, pap smears, and needle biopsies. The assay can be applied to samples of cells or biological fluid for direct detection of lipids without performing a lipid extraction. A kit designed for use in a clinical setting can use either an ELISA or fluorogenic format, and would be similar to that designed for use in a research lab.

Another embodiment of the present invention involves a rapid plate-based assay kit for a lipid phosphatase activity, including lipids, plates, and detection reagents. Such kits will satisfy particular needs from clinical or research laboratory scientists, for example. A rapid plate-based assay apparatus can be used for HTS drug discovery efforts in the pharmaceutical industry.

PTEN and SHIP2 are important targets for anti-cancer and anti-diabetic drug development, and there is a need in the pharmaceutical industry for new methods of screening for lipid phosphatase inhibitors or enhancers. In-vitro assays of the present invention are advantageous for the discovery of potential drugs targeting lipid phosphatases. Present methods involve costly and cumbersome radioactive extractions, while the present assay formats provide a less expensive, simple, and non-radioactive alternative. The present assays for use in drug discovery are convertible to a HTS format. The amplified luminescence homogeneous proximity assay (ALPHA) and the fluorogenic assay formats are particularly well-suited for HTS applications. Automation of assay platforms can be performed using an automated liquid handling system interfaced to one of several micro plate readers. The system includes a multi-well pipettor/washer integrated with a plate-handling robot for highly accurate and simultaneous delivery of microvolumes of liquids into wells of a large number of micro plates, including 96-, 384-, and 1 533-well densities. Transfer to the plate reader, sample analysis, and data collection is also automated and computer controlled.

The preceding description has been presented only to illustrate and describe the invention. The preferred embodiment was chosen and described in order to best explain the principles of the invention and its practical application. The preceding description is intended to enable others skilled in the art to best utilize the invention in various embodiments and with various modifications as suited to the particular use contemplated. It will be apparent to those of ordinary skill in the art that numerous modifications can be made without departing from the principles and concepts of the invention as set forth in the claims.

What is claimed is:

1. A lipid phosphatase assay method comprising the steps of: exposing a lipid detector protein containing a lipid recognition motif with a binding specificity for a product lipid of a lipid phosphatase, to a solution containing a substrate lipid of said lipid phosphatase; and
determining whether said product lipid is present in said solution.

2. The lipid phosphatase assay method according to claim 1, wherein the assay is a direct assay or a competitive assay wherein said product lipid has a stronger affinity to said lipid detector protein than said substrate lipid.

3. The lipid phosphatase assay method according to claim 1, wherein said lipid detector protein is an antibody against said product lipid or a lipid recognition protein(LRP) with specificity for said product lipid.

4. The lipid phosphatase assay method according to claim 3, wherein said lipid recognition protein contains an affinity tag fusion with PH or other lipid-binding domains.

5. The lipid phosphatase assay method according to claim 1, wherein said assay is a plate-based assay.

6. The lipid phosphatase assay method according to claim 5, wherein said assay is an enzyme linked immunosorbent assay (ELISA).

7. The lipid phosphatase assay method according to claim 1, further comprises: prior to exposing said lipid detector protein to the solution, coating a substrate of an assay plate with a non-radioactively labeled substrate lipid.

8. The lipid phosphatase assay method according to claim 7, wherein said assay plate is coated with streptavidin, glutathione or Protein A.

9. The lipid phosphatase assay method according to claim 1, wherein said assay is an amplified luminescence proximity homogenous assay (ALPHA).

10. The lipid phosphatase assay method according to claim 1, wherein said assay is a fluorogenic assay.

11. The lipid phosphatase assay method according to claim 10, wherein the assay is a fluorescence polarization(FP) assay, fluorescence resonance energy transfer(FRET) assay or time-resolved fluorescence resonance energy transfer(TR-FRET) assay.

12. The lipid phosphatase assay method according to claim 1, wherein additional lipids are present in said solution.

13. The lipid phosphatase assay method according to claim 1, wherein said lipid phosphatase acts on any PIPn and is a member selected from the group consisting of SHIP1, SHIP2, PTEN, PTPRQ, SKIP, Myotubularin, MTMR2 and OCRL1.

14. The lipid phosphatase assay method according to claim 1, wherein said substrate lipid is PI(3,4,5)P₃, PI(3,4)P₂, PI(3,5)P₂, PI(4,5)P₂, PI(3)P, PI(4)P, or PI(5)P.

15. The lipid phosphatase assay method according to claim 1, wherein said product lipid is PI(3,4)P₂, PI(4,5)P₂, PI(3,5)P₂, PI(3)P, PI(4)P, PI(5)P, or Phosphatidyl Inositol.

16. A lipid phosphatase assay kit comprising: a lipid detector protein containing a lipid recognition motif with a binding specificity for a product lipid of a lipid phosphatase, and a solution containing a substrate lipid of said lipid phosphatase.

17. The lipid phosphatase assay kit according to claim 16, wherein said assay kit is a direct assay kit or a competitive assay kit wherein said product lipid has a stronger affinity to said lipid detector protein than said substrate lipid.

18. The lipid phosphatase assay kit according to claim 16, further comprising a multi-well assay plate.

19. The lipid phosphatase assay kit according to claim 18, wherein said substrate lipid is non-radioactively labeled and is immunobilized in wells of said multi-well assay plate.

20. A lipid phosphatase assay kit according to claim 16, wherein said assay kit is used for detection of said target lipid in bodily tissue, blood, and serum samples.

21. The lipid phosphatase assay kit according to claim 16, wherein said lipid detector protein is an antibody against said product lipid or a lipid recognition protein(LRP) with specificity for said product lipid.

22. The lipid phosphatase assay kit according to claim 21, wherein said lipid recognition protein contains an affinity tag fusion with PH or other lipid-binding domains.

23. The lipid phosphatase assay kit according to claim 18, wherein said assay is an enzyme linked immunosorbent assay (ELISA).

24. The lipid phosphatase assay kit according to claim 18, wherein said substrate lipid is immobilized in wells of said multi-well assay plate.

25. The lipid phosphatase assay kit according to claim 18, wherein said assay plate is coated with streptavidin, glutathione or Protein A.

26. The lipid phosphatase assay kit according to claim 16, wherein said assay is an amplified luminescence proximity homogenous assay (ALPHA).

27. The lipid phosphatase assay kit according to claim 16, wherein said assay is a fluorogenic assay selected from the group consisting of a fluorescence polarization (FP) assay, a fluorescence resonance energy transfer(FRET) assay and a time-resolved fluorescence resonance energy transfer(TR-FRET) assay.

28. The lipid phosphatase assay kit according to claim 16, wherein additional lipids are present in said solution.

29. The lipid phosphatase assay kit according to claim 16, wherein said lipid phosphatase acts on any PIPn and is a member selected from the group consisting of SHIP1, SHIP2, PTEN, PTPRQ, SKIP, Myotubularin, MTMR2 and OCRL1.

30. The lipid phosphatase assay kit according to claim 16, wherein said substrate lipid is PI(3,4,5)P₃, PI(3,4)P₂, PI(3,5)P₂, PI(4,5)P₂, PI(3)P, PI(4)P, or PI(5)P.

31. The lipid phosphatase assay kit according to claim 16, wherein said product lipid is PI(3,4)P₂, PI(4,5)P₂, PI(3,5)P₂, PI(3)P, PI(4)P, PI(5)P, or Phosphatidyl Inositol.

32. A method for screening a disease caused alteration of a lipid phosphatase comprising the step of using the lipid phosphatase assay method of claim 1 to detect changes in the lipid phosphatase activity in bodily tissue, blood, or serum samples.

33. The method of claim 32, wherein the disease is non-insulin dependant, Type II diabetes.

34. The method of claim 32, wherein the disease is Cowden's disease or cancer.

35. A method for screening a disease caused alteration of a lipid phosphatase comprising the step of using the lipid phosphatase assay kit of claim 16 to detect changes in the lipid phosphatase activity in bodily tissue, blood, or serum samples.

36. The method of claim 35, wherein the disease is non-insulin dependant, Type II diabetes.

37. The method of claim 35, wherein the disease is Cowden's disease or cancer.

38. A method for screening a compound having an enhancing or inhibiting effect on a lipid phosphatase comprising the step of using the lipid phosphatase assay method of claim 1 to detect changes in the lipid phosphatase activity.

39. A method for screening a compound having an enhancing or inhibiting effect on a lipid phosphatase comprising the step of using the lipid phosphatase assay kit of claim 16 to detect changes in the lipid phosphatase activity.
